(19) United States
(12) Patent Application Publication
(10) Pub. No.: US 2008/0198872 A1
(43) Pub. Date: Aug. 21, 2008

(54) Ultrasonic Diagnostic Imaging System With Multiplexed Voice and Image Communication

(75) Inventor: Michael Pierce, Carnation, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/914,274

(22) PCT Filed: May 10, 2006
(86) PCT No.: PCT/IB06/51476

§ 371 Date: Nov. 13, 2007

Related U.S. Application Data
(60) Provisional application No. 60/683,508, filed on May 19, 2005.

(51) Int. Cl. H04L 29/06; H04L 12/50; A61B 8/00

(52) U.S. Cl. 370/465; 370/466; 709/230; 600/437; 370/401

(57) ABSTRACT

An ultrasound system which is capable of sending images and/or reports over a data network by means of an Internet protocol has a sound card coupled to a microphone and a loudspeaker. When operator of the ultrasound system speaks into the microphone the voice is digitized by the sound card and the voice data is packaged as payloads of data packets. The packets are sent over the same data network by a protocol stack using an Internet protocol. The packets are received and returned to analog voice signals at a receiving terminal. The voice capability can reach other terminals on the network or external correspondents by means of the Internet or external networks such as public switched telephone networks.

This invention relates to medical diagnostic ultrasound systems and, in particular, to ultrasonic diagnostic imaging systems capable of multiplexing voice and image information over a common data network.

At many medical facilities it is common practice for patients to be scanned in an ultrasound exam by a sonographer and for the images to be read for diagnosis by a radiologist or echocardiographer in a separate reading room or at a remote workstation. In such a setting the physician reading the images can make diagnoses of multiple patients being scanned at the same time through the networking of the ultrasound systems used for the examinations with the reading workstation. When the diagnoses are being made while the patient is in the scanning room, a physician may frequently learn that additional images or different views would be helpful or necessary for a reliable diagnosis. At those times the diagnosing physician will want the additional scanning to be performed while the patient is still available in the medical facility. The conventional way this is done is for the physician to leave the reading room and go to the scanning room to try to intercept the patient and the sonographer before the patient has departed. Alternatively, the physician may try to do this by telephoning the sonographer in the scanning room. It would be desirable to be able to contact the sonographer more quickly and easily from the reading room.

US patent application publication no. 2003/0083563 (Katsman et al.) provides one solution to this situation, which is to enable the sonographer and the physician to communicate with each other through the ultrasound system. The ultrasound system and the reading workstation are both equipped with a microphone, loudspeaker, and a speech recognition and processing system. When a person speaks into the microphone the speech is converted into digital speech data and compressed. The compressed speech data is transmitted over the network connecting the two devices to the terminal. The receiving terminal decompresses the data, the speech recognition and processing system processes the digital speech data and transmits it to the loudspeaker. By this means the sonographer and the reading physician can speak to each other and the physician can give instructions to the sonographer during the ultrasound exam. However the manner in which the image and voice data share the network connection is not explained. It would be desirable to multiplex the voice and image communications so that the voice and image data would automatically share the network connection whenever a speaker decides to speak. It is further desirable to be able to extend the ability to engage in such voice and image communication to communicating with other people not on the medical facility's network.

In accordance with the principles of the present invention, a diagnostic ultrasound system and remote terminal are described which are able to exchange voice communication through packets of voice data using a TCP/IP Internet protocol. When image communication between the same two devices also uses a TCP/IP protocol, the image and voice data packets can both share the same data network, with the header information of the packets providing the correct and accurate routing of the respective data packets. The packetized voice transmissions can be routed to others outside the local area network over external carrier system such as public telephone networks. An embodiment of the present invention can thus also be used to communicate with people outside of the medical facility. A real-time protocol can be used to ensure that transmitted voice packets are received in a timely way so as to be reproduced as normal, uninterrupted speech.

In the drawings:

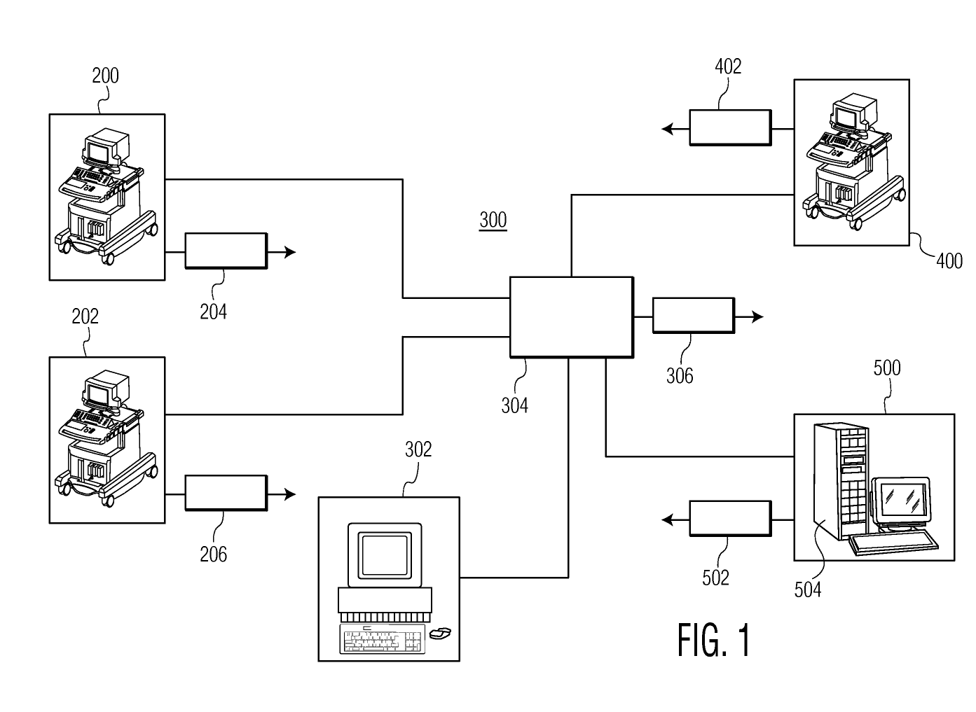

FIG. 1 illustrates a medical network including a plurality of ultrasound systems and a diagnostic workstation constructed in accordance with the principles of the present invention.

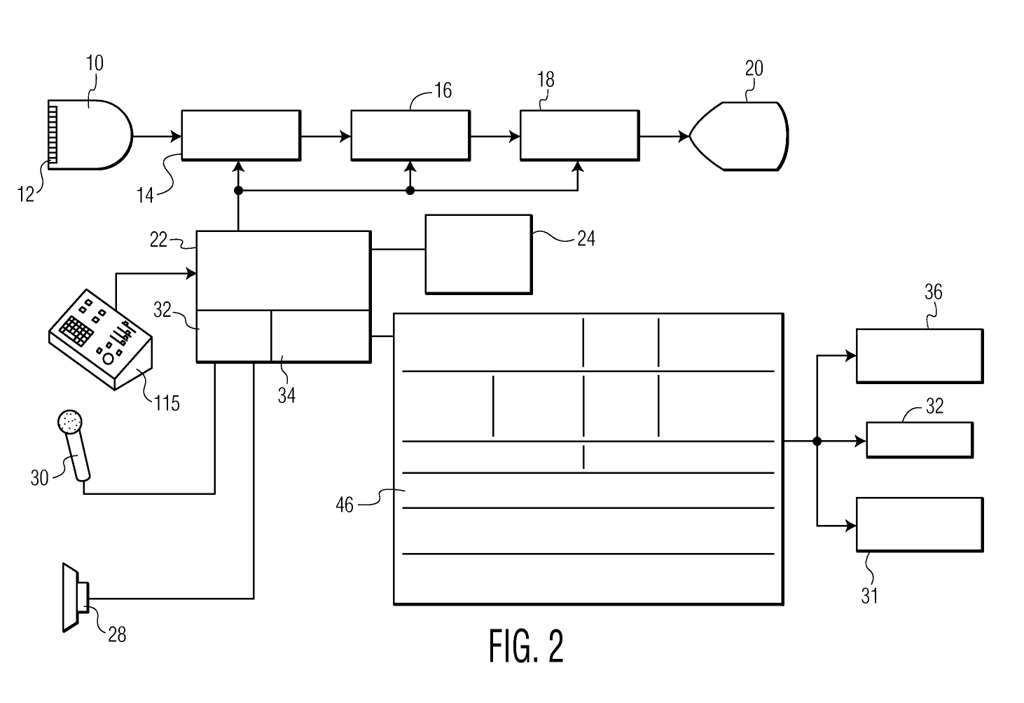

FIG. 2 illustrates in block diagram form the details of a voice and data messaging ultrasound system constructed in accordance with the principles of the present invention.

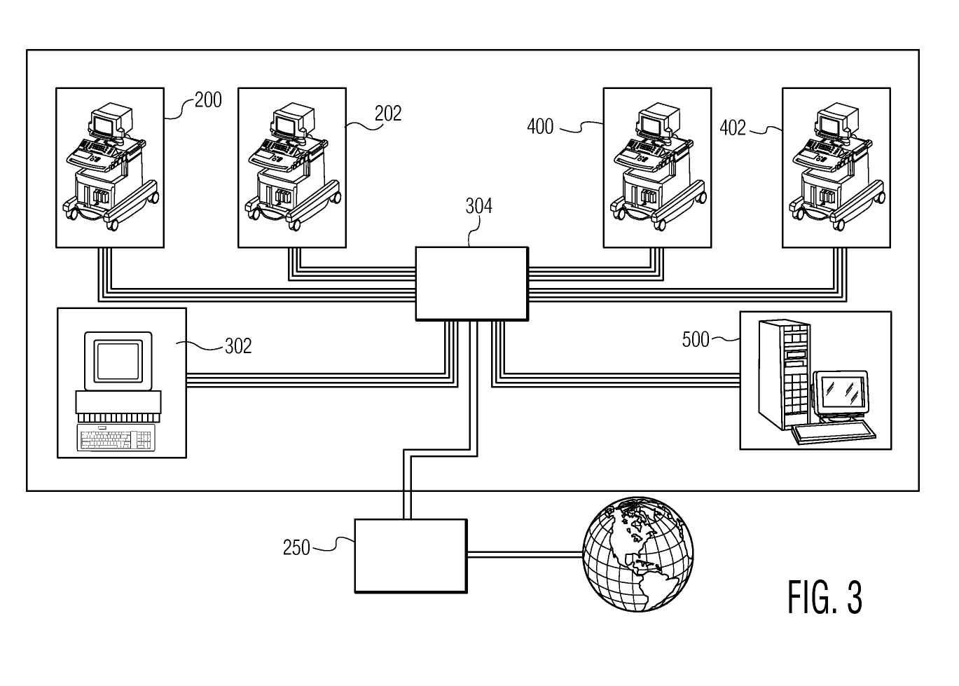

FIG. 3 illustrates an ultrasound network of another embodiment of the present invention in which voice communication may be conducted from an ultrasound system over a public switched telephone network or the Internet.

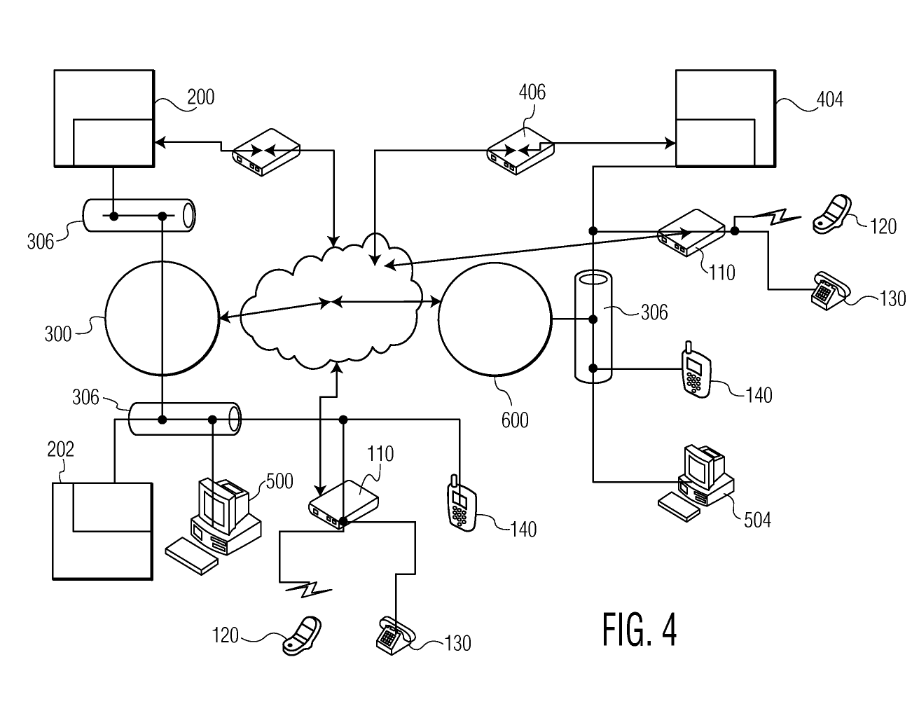

FIG. 4 illustrates another network embodiment of the present invention which shows the variety of devices with which voice communication may be had in accordance with the principles of the present invention.

Referring first to FIG. 1, a medical packet switching network 300 includes several ultrasound systems 200, 202 and 400 networked together by a hub 304 such as a router. Also connected to the network 300 are a diagnostic workstation 302 at which a physician can read and make diagnoses from ultrasound images acquired from patients by the ultrasound systems 200, 202 and 300. Images and reports are routed from the ultrasound systems to the workstation in packets of data using a TCP/IP protocol. Each device on the network has a local IP address which is used to identify the device on the network to TCP/IP packet traffic. Also connected to the network 300 is a terminal 500 including a desktop PC 500. The desktop PC may be a physician's office computer, for instance. The terminal 500 can likewise send and receive packetized data over the network 300. In addition to the network Ethernet connections each of the ultrasound systems and the office PC are also shown with modems 204, 206, 402 and 502 by which these devices can connect to external devices and networks such as the Internet. The ultrasound systems 200, 202 and 400 on the network 300 as well as the workstation 302 and the desktop PC 504 can send and receive images and reports using a TCP/IP protocol as described in U.S. Pat. No. 5,715,823 (Wood et al.) Electronic messaging between and among these systems is also possible as described in U.S. Pat. No. 5,897,498 (Canfield, II et al.)

In accordance with the principles of the present invention each of the ultrasound systems, the workstation and the office PC are capable of providing voice communication between operators of the devices over the same packet switching data network 300. An embodiment of an ultrasound system with these capabilities is shown in FIG. 2. At the top of the drawing is the ultrasound signal path of the system, including a probe 10 with an array transducer 12 which transmits and receives ultrasound signals, a beamformer 14 which provides steering and focusing of transmit beams and processes echo signals received by the elements of the array transducer to form coherent echo signals, an ultrasound signal processor 16, an image processor 18, and a display 20 on which the ultrasound image and data are displayed. The operation of these components is coordinated by a system controller 22. The operation of the ultrasound system is directed by operator controls 115 coupled to the system controller. The system controller 22 can store images and diagnostic reports produced by the ultrasound system on storage device 24. A microphone 30 and a loudspeaker 28 (which may be separate or part of a common headset) are provided on the ultrasound system to enable the operator to communicate by voice with people at other devices on the network 300 and, as discussed below, at remote locations. Ultrasound systems have long had loudspeakers for the reproduction of audio Doppler, and systems such as the Philip iU22 ultrasound system have recently been equipped with microphones for voice control of the system. The microphone 30 and the loudspeaker 28 are coupled to an input and an output of a sound card 32. When the operator speaks into the microphone his or her voice is digitized by an A/D converter on the sound card. For voice control of the ultrasound system the converted voice signal is processed by voice recognition software and the output used to control the system. In accordance with the principles of the present invention the digitized voice signals are sent over the packet switching network 300 and received as voice output by a loudspeaker 28 of another device on the system. This is done by an operating system 34 which runs communication software including execution of a voice communication protocol such as that illustrated by protocol stack 46.

From an overall viewpoint, the operator's voice is digitized by the sound card into bytes of data. A nominal voice bandwidth is 4 kHz, which means that a sampling bandwidth of 8 kHz would be sufficient to digitize the typical voice frequencies. Most sound cards are capable of digitizing analog signals at a much higher rate, usually on the order of 44 kHz sampling to produce 16-bit bytes. Since the voice bandwidth does not require this high a digitization rate, a number of successive bytes can be aggregated and sent as the payload of an IP packet. In addition, the digitized voice data may be compressed before transmission using a compression protocol such as MP-MLQ or ACELP, Standard ITU-T G.723.1. The packetized voice data is then sent from the host ultrasound system over the network. This may be done directly from one endpoint to another, e.g., from the ultrasound system directly to the workstation, but generally the packet traffic is mediated by a gatekeeper such as a router which manages data traffic by performing duties such as translating IP addresses of the endpoint devices, granting or denying access, call signaling to connect the call, call authorization, bandwidth management and call management. The voice packets may be directed by multiple gatekeepers before reaching the destination device. At the receiving device the packet data is unpacked in accordance with instructions provided by the packet protocols and reassembled to its original state. The bytes of data are converted back to analog signals by a D/A converter in the sound card at the receiving endpoint and played as a voice through the loudspeaker at the receiving end.

The protocol stack 46 shown is typical for the H.323 standard for voice communication over a TCP/IP network. Other protocols such as SIP (Session Initiation Protocol) may alternatively be used. At the bottom of the stack is the physical layer which performs connection services and signal conversion for the data link layer above. The data link layer in this embodiment is an Ethernet protocol layer. The network layer is the IP protocol so that the voice packets can share the communication medium with other IP service packets including image communication between the ultrasound system and the workstation. At the next layer it is seen that the audio and registration packets use the User Datagram Protocol (UDP) while the control and signaling packets use the Transmission Control Protocol (TCP) as the transport protocol. Both the source and receiver endpoints support the H.245 and Q.931 protocols. H.245 allows usage of channels and Q.931 is needed for call signaling and setting up the call. In the illustrated stack H.225.0/Q.931 Call Signaling is used to provide the signaling for call control. For the received voice to sound natural and not broken up, it is important for the voice data to arrive at the destination substantially in real time. This is accomplished by the use of RTP, the real time transport protocol that carries the voice packets. When the call is made through a gatekeeper (e.g., a router) rather than directly from endpoint to endpoint as is possible in a single LAN (Local Area Network) with direct endpoint call signaling between the two transport addresses, the H.225 RAS (Registration, Admission, Status) channel is used to communicate between endpoints and the gatekeeper. The RAS channel performs procedures such as determining a gatekeeper with which it should register, endpoint registration of the packet's transport and alias (alternate) addresses, endpoint location, and admission, status, and disengage messages. The procedure to set up a call involves discovering a gatekeeper with which the endpoint can register; registration with the gatekeeper; entering the call setup phase; capability exchange between the endpoint and the gatekeeper; and establishing the call. In this example the voice packet is sent by way of the Ethernet connection 36, although communication may also be delivered and received by other ports such as a modem 32 or a serial port 31.

By use of this protocol stack a voice packet is passed from the source terminal, the ultrasound system in this instance, to a series of one or more gatekeepers (routers) until finally arriving at the destination terminal, the workstation in this example. At the workstation the various header layers are examined and stripped off until the voice data is delivered to the sound card, where it is converted to an analog signal and played through the loudspeaker 28 at the workstation. A codec may be used to decompress data that was compressed at the source. The workstation has the same communication hardware, software and protocol stack as does the ultrasound system so that the physician at the workstation can communicate by voice back to the ultrasound system operator.

In a constructed embodiment the operating system 34 will generally run user interface software to permit the ultrasound system or workstation operator to easily access the voice communication capability. For calling out, such software will display a selection of IP addresses or other alias addresses such as telephone numbers from which the operator can choose to initiate a call. When an incoming call is received, the software will make an audible sound through the loudspeaker 28 and/or display an incoming call icon on the display screen. The operator will touch a key on the control panel 115 or on the display screen to answer the call.

An embodiment of the present invention need not be constrained to calling only those connected to the LAN of the medical facility. The same voice packets can be transmitted by a gateway 250 which is connected to the Internet or a public switched telephone network as illustrated in FIG. 3. This compatibility with TCP/IP and IP addressing enables communication with other terminals and telephones capable of dealing with voice data in the form of IP packets. An operator at an ultrasound system can thus call a physician at home or at a remote office by this capability.

FIG. 4 illustrates some of the communication possibilities presented by the present invention. Voice communication may be conducted between operators of ultrasound systems 200 and 202 over their local network 300 through Ethernet connections 306 and with the operator of the workstation 500. The can talk with others outside of the local network 300 over the Internet, such as the operator of ultrasound system 404 at another location. Connections can be made either through the local networks 300 and 600 or through cable/DSL/satellite modems 204 and 406. The voice communications can be received by telephones 140 with Internet voice capabilities and by conventional mobile telephones 120 and land line telephones 130 which have voice-over-Internet phone adapters 110.

What is claimed is:

1. An ultrasound system which can send and receive images or reports over a data network by an Internet protocol comprising:
a loudspeaker;
a microphone;
a digitizing circuit coupled to the microphone to digitize voice signals; and
a communication protocol, responsive to digitized voice signals, which acts to transmit and/or receive packets of voice data using an Internet protocol.

2. The ultrasound system of claim 1, wherein the ultrasound system transmit and/or receives packets of voice data to recreate substantially real time speech.

3. The ultrasound system of claim 1, further comprising:
a data storage device which stores images or reports produced by the ultrasound system,
wherein the communication protocol transmits and/or receives packets of voice data over the same data network as that over which images or reports are sent or received.

4. The ultrasound system of claim 1, wherein the network comprises a local area network; and
wherein the packets of voice data include the IP addresses of the source and destination devices on the local area network.

5. The ultrasound system of claim 1, wherein the ultrasound system is coupled to a local area network; and
wherein the destination device of transmitted voice data packets is not a device on the local area network.

6. The ultrasound system of claim 5, wherein the transmitted voice data packets are transmitted over a public switched telephone network.

7. The ultrasound system of claim 5, wherein the transmitted voice data packets are transmitted over the Internet.

8. The ultrasound system of claim 1, wherein the packets of voice data are transmitted and/or received directly between the ultrasound system and another endpoint device.

9. The ultrasound system of claim 1, wherein the packets of voice data are mediated by one or more routers during transport between the ultrasound system and another endpoint device.

10. A method of transmitting voice communication and diagnostic images between an operator of an ultrasound system and a diagnostic image reader located at a computer terminal coupled to the ultrasound system by a data network, comprising:
producing a diagnostic image on the ultrasound system;
transmitting the diagnostic image over the data network in one or more data packets to the computer terminal using an Internet protocol;
speaking into a microphone;
digitizing voice signals;
transmitting the digitized voice signals over the data network in one or more data packets to a destination device using an Internet protocol; and
reproducing the voice through a loudspeaker on the destination device.

11. The method of claim 10, wherein transmitting digitized voice signals further comprises transmitting the voice of the ultrasound system operator to the computer terminal and transmitting the voice of the diagnostic image reader to the ultrasound system to reproduce real time conversation.

12. The method of claim 10, further comprising producing a diagnostic image by the ultrasound system in response to voice communication by the diagnostic image reader.

13. The method of claim 10, wherein the computer terminal comprises a diagnostic image analysis workstation.

14. The method of claim 13, wherein the data network comprises a local area network to which the diagnostic image analysis workstation and a plurality of ultrasound systems are coupled;
wherein each ultrasound system and workstation has a unique IP address on the network; and
wherein transmitting digitized voice signals further comprises addressing a voice data packet to the IP address of a destination device at which voice is to be reproduced.

15. The method of claim 14, wherein transmitting further comprises receiving a digitized voice packet at a router on the network; and
retransmitting the digitized voice packet to a destination device.

16. The method of claim 10, wherein transmitting further comprises transmitting the digitized voice packet to a gateway; and
retransmitting the digitized voice packet from the gateway to a destination device.

17. The method of claim 10, wherein the data network includes a public switched telephone network.

18. The method of claim 10, wherein transmitting further comprises utilizing a TCP/IP protocol.

19. The method of claim 18, wherein transmitting the digitized voice signals further comprises utilizing the TCP and UDP protocols.
